(19) United States
(12) Patent Application Publication
(10) Pub. No.: US 2025/0114295 A1
(43) Pub. Date: Apr. 10, 2025

(54) COMPOSITIONS SUITABLE FOR USE IN SKIN CARE

(71) Applicant: JOHNSON & JOHNSON CONSUMER INC., Skillman, NJ (US)

(72) Inventors: Ramine PARSA, Lawrenceville, NJ (US); Peter LYTE, Princeton, NJ (US); Wen-Hwa Ting LI, Cranbury, NJ (US)

(21) Appl. No.: 18/905,672

(22) Filed: Oct. 3, 2024

Related U.S. Application Data
(60) Provisional application No. 63/588,036, filed on Oct. 5, 2023.

(51) Int. Cl. A61K 8/9789; A61K 8/9794; A61Q 17/00; A61Q 19/08

(52) U.S. Cl. CPC A61K 8/9789; A61K 8/9794; A61Q 17/00; A61Q 19/08

(57) ABSTRACT

The invention relates to composition for skin care. In particular the invention relates to skincare composition comprising a myrrh resin extract, and a processed oat ingredient. The invention also relates to a method of treating the skin using said composition, and to a method for activating lipid production in the skin layers.

### CROSS-REFERENCE TO RELATED APPLICATIONS

This application claims the benefit of U.S. provisional application 63/588,036 filed on Oct. 5, 2023, the complete disclosure of which is hereby incorporated herein by reference for all purposes.

### FIELD

The invention generally relates to compositions for skin care. In particular the invention relates to skincare compositions comprising a myrrh resin extract, and a processed oat ingredient. The invention also relates to a method of treating the skin using said composition.

### BACKGROUND

The human skin is subject to certain aging processes, some of which are attributable to intrinsic processes (e.g. chronoaging) and some of which are attributable to exogenous factors (e.g. photo-aging). In addition, temporary or even lasting changes to the skin can occur, such as acne, greasy or dry skin, keratoses, rosacea, light-sensitive, inflammatory, erythematous, and allergic or autoimmune-reactive reactions, such as dermatosis and photodermatosis.

The consequences of the above-mentioned aging processes can include thinning of the skin, weaker interlacing of epidermis and dermis, and a reduction in the number of cells and the supplying blood vessels. These consequences are often undesirable, and individuals suffering from these issues will look to topical treatments to address them.

Oat lipids are known to have an anti-inflammatory effect and possess dual PPARa and PPARb/d agonistic activities; Oat lipids also induce keratinocyte differentiation and ceramide synthesis in primary human keratinocytes, which may provide skin barrier improvement (Su-Hyoun Chon et al., Experimental Dermatology, 2015, 24, 290-295).

Colloidal oat has high lipid content (3-18%) compared to other cereal grains, and the lipid fraction, oat oil, is enriched with unsaturated fatty acids (Banas A et al. J Exp Bot 2007:58:2463-2470). Peroxisome proliferator-activated receptors (PPARs) are ligand activated nuclear receptors, regulating lipid homoeostasis in our bodies. In skin, PPAR receptor activation is known to improve epidermal barrier functions, exert anti-inflammatory effects and expedite the wound healing process (Schmuth M et al. J Lipid Res 2008:49:499-509.)

Myrrh resin is a natural extract from tree species from the genus Commiphora. Myrrh resin is known since ancient times for its application in perfume and medicine, for example as an antiseptic. Topical application of myrrh resin on the skin can cause skin irritation.

Treating skin conditions associated with aging, such as skin atrophy, acne, photo-aging, and in reducing the appearance of wrinkles, fine lines, stretch marks, or cellulite is challenging. Consumers are careful about the product they apply topically and show an increased interest for active ingredients of natural origin.

Accordingly, there is a need to provide natural extracts that can improve epidermal barrier functions and provide an anti-inflammatory effect. Improving the potentiality of known and trusted natural ingredients which are efficacious but also gentle enough on the skin to be well-tolerated is particularly desirable.

### SUMMARY

One aspect of the invention pertains to skincare composition comprising a myrrh resin extract, and a processed oat ingredient.

This aspect may be combined with a variety of embodiments, in any combination. Thus, in some embodiments the myrrh resin extract is produced from the resin of a plant from the genus Commiphora. In one or more embodiments, the myrrh resin extract is obtained by extraction with an organic solvent. In some embodiments, the myrrh resin extract is present in an amount ranging from about 0.000001% to about 10% by total weight of the composition. In one or more embodiments, the processed oat ingredient is selected from the group consisting of fermented oat, colloidal oat, oat extract, oat oil, and combinations thereof. In one or more embodiments, the processed oat ingredient is present in an amount ranging from about 0.001% to about 10% by total weight of the composition. In one or more embodiments, the weight ratio of myrrh resin extract to processed oat ingredient is from about 10:1 to about 1:1000. In some embodiments, the skincare composition further comprises an ingredient selected from the group consisting of surfactants, chelating agents, emollients, humectants, conditioners, preservatives, opacifiers, fragrances, and combinations of two or more thereof. In one or more embodiments, the skincare composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

Another aspect of the invention pertains to a skincare composition comprising about 0.005 wt. % to about 1.5 wt. % myrrh resin extract by total weight of the composition; and about 0.1 wt. % to about 2.5 wt. % by total weight of the composition of a processed oat ingredient selected from the group consisting of oat oil.

Another aspect of the invention pertains to a method of skin treatment comprising topically applying to the skin a composition according to any of the embodiments described above.

Another aspect of the invention pertains to a method of treating signs of skin aging, comprising topically applying to the skin in need of treatment for skin aging a skincare composition according to any of the embodiments described above.

Another aspect of the invention pertains to a method for activating lipid production in the skin layers of a skin in need thereof, comprising topically applying to the said skin a skincare composition comprising a myrrh resin extract and a processed oat ingredient.

Another aspect of the invention pertains to a method for activating ceramide production in the skin layers of a skin in need thereof, comprising topically applying to the said skin a skincare composition comprising a myrrh resin extract and a processed oat ingredient.

This aspect may be combined with a variety of embodiments, in any combination. Thus, in some embodiments of the method disclosed above, the processed oat ingredient is selected from the group consisting of fermented oat, colloidal oat, oat extract, oat oil, and combinations thereof. In one or more embodiments the processed oat ingredient is present in an amount ranging from about 0.001% to about 10% by total weight of the skincare composition. In one or more embodiments the myrrh resin extract is present in an amount ranging from about 0.000001% to about 10% by total weight of the composition. In one or more embodiments the skincare composition topically applied to the skin comprises: about 0.005 wt. % to about 1.5 wt. % myrrh resin extract by total weight of the composition; and about 0.1 wt. % to about 2.5 wt. % by total weight of the composition of a processed oat ingredient selected from the group consisting of oat oil.

Another aspect of the invention pertains to a method for boosting skin Barrier by stimulating skin barrier genes expression, for example Involucrin, Filaggrin and Loricrin expressions, in the skin layers of a skin in need thereof, comprising topically applying to the said skin a skincare composition comprising a myrrh resin extract and a processed oat ingredient.

Another aspect of the invention pertains to a method for boosting the anti-aging effects of processed oat in the skin layers of a skin in need thereof, comprising topically applying to skin a skincare composition comprising a myrrh resin extract and a processed oat ingredient preferably selected from the group consisting of fermented oat, colloidal oat, oat extract, oat oil, and combinations thereof.

These and other features and advantages of the present invention will be readily apparent from the following detailed description of the invention.

### DETAILED DESCRIPTION

As used herein, a skin care product or skin care composition is a product that can be applied to the skin or hair of a user, providing a benefit to that user. The benefit can be, for example, hydration of the skin or hair or delivery of one or more therapeutic or benefit agents to the skin or hair, as described in more detail below.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The general convention in the scientific and technical literature is applied: the last decimal place of a numerical value indicates its degree of accuracy. Where no other error margins are given, the maximum margin is ascertained by applying the rounding-off convention to the last decimal place, for example for a measurement of 3.5%, the error margin is 3.45-3.54.

All percentages (%) are by weight unless otherwise specified herein.

One aspect of the invention pertains to a skincare composition comprising:
a. a myrrh resin extract, and
b. a processed oat ingredient.

Surprisingly this combination of myrrh resin extract and processed oat ingredient has been found provide an increase in the ceramide production and an increase in the expression of genes coding for skin barrier proteins, without the drawback such as skin irritation traditionally associated with natural extracts such as Myrrh resin.

This increase in ceramide and in skin barrier proteins is believed to provide a beneficial effect to the skin; for example, to reduce the signs of aging of the skin.

By “extract” it is meant the substance, or the mixture of substances that is withdrawn by a chemical process from the myrrh resin. The chemical process may be for example the treatment of the myrrh resin with a solvent so as to remove a soluble substance, or a mixture of soluble substances, from the myrrh resin. The “myrrh resin extract” is thus a subset of the myrrh resin.

By “processed” it is meant that the raw ingredient, for example Oat, has been submitted to industrial treatment or process; to isolate a fraction of the raw ingredient constituents, or to modify the properties of the raw ingredient. It may be for example a fermentation or the extraction by chemical or physical means of an oil or other specific compounds.

In one or more embodiments, the skincare composition may comprise a myrrh resin extract which is produced from the resin of a plant from the genus Commiphora.

Preferably, from the resin of plants such as Commiphora Myrrha, or Commiphora molmol.

In this embodiment by “produced” it is meant that the myrrh resin is a secretion obtained from the plant bark.

In one or more embodiments, the myrrh resin extract is obtained by extraction with an organic solvent.

Myrrh resin is a complex mixture of chemical compounds, and it has been discovered that the fraction of myrrh resin isolated by an extraction with an organic solvent, such as a polar organic solvent, provides beneficial effects according to the invention.

Preferably the organic extraction of myrrh resin is performed with an alcoholic solvent such as such as methanol, ethanol, propanol, isopropanol, butanol; preferably ethanol.

The resin produced by the bark of a tree from the Commiphora genus is processed with an organic solvent, such as ethanol, to give a fraction of the said resin that is designated as “myrrh resin extract” according to the present invention.

The myrrh resin extract may also be blended with Triethyl acetylcitrate, for example the myrrh resin extract may contain from about 10% to about 20% Triethyl acetylcitrate, preferably from about 12% to about 15%.

In one or more embodiments, the myrrh resin extract is present in an amount ranging from about 0.000001% to about 10% by total weight of the composition.

Preferably the myrrh resin extract is present in an amount ranging from about 0.000001% to about 1% by total weight of the composition; or about 0.000001% to about 0.5% by total weight of the composition; or about 0.000001% to about 0.2% by total weight of the composition; or about 0.000001% to about 0.1% by total weight of the composition; or about 0.000001% to about 0.02% by total weight of the composition, or about 0.00001% to about 0.02% by total weight of the composition, or about 0.0001% to about 0.02% by total weight of the composition, or about 0.001% to about 0.02% by total weight of the composition, or about 0.005% to about 1.5% by total weight of the composition, or about 0.005% to about 0.1% by total weight of the composition, or about 0.005% to about 0.015% by total weight of the composition. Preferably the myrrh resin extract is present in an amount above 0.001% by total weight of the composition. Preferably the myrrh resin extract is present in an amount above 0.005% by total weight of the composition. Preferably the myrrh resin extract is present in an amount above 0.01% by total weight of the composition.

Preferably the myrrh resin extract is present in an amount below 1% by total weight of the composition. Preferably the myrrh resin extract is present in an amount below 0.5% by total weight of the composition. Preferably the myrrh resin extract is present in an amount below 0.2% by total weight of the composition. Preferably the myrrh resin extract is present in an amount below 0.1% by total weight of the composition. Preferably the myrrh resin extract is present in an amount below 0.05% by total weight of the composition. Preferably the myrrh resin extract is present in an amount below 0.02% by total weight of the composition.

Preferably the myrrh resin extract is not encapsulated.

In one or more embodiments, the processed oat ingredient is selected from the group consisting of fermented oat, colloidal oat, oat extract, oat oil, and combinations thereof.

In a preferred embodiment, the processed oat ingredient is an oat oil.

In a preferred embodiment the processed oat ingredient is a lipid fraction obtained from oat (Avena Sativa). This lipid fraction may be obtained by any technical mean known to the person skilled in the art.

In one or more embodiments, the processed oat ingredient is present in an amount ranging from about 0.001% to about 10% by total weight of the composition.

Preferably the processed oat ingredient is present in an amount ranging from about 0.001% to about 5%; or from about 0.001% to about 1%; from about 0.001% to about 0.5%. Preferably the processed oat ingredient is present in an amount above 0.005% by total weight of the composition. Preferably the processed oat ingredient is present in an amount above 0.01% by total weight of the composition. Preferably the processed oat ingredient is present in an amount above 0.05% by total weight of the composition. Preferably the processed oat ingredient is present in an amount above 0.1% by total weight of the composition. Preferably the processed oat ingredient is present in an amount below 5% by total weight of the composition. Preferably the processed oat ingredient is present in an amount below 1% by total weight of the composition. Preferably the processed oat ingredient is present in an amount below 0.5% by total weight of the composition

In one or more embodiments, the weight ratio of myrrh resin extract to processed oat ingredient is from about 10:1 to about 1:1000.

In a preferred embodiment, the weight ratio of myrrh resin extract to processed oat ingredient is from about 1:1 to about 1:100; preferably from about 1:10 to about 1:80; preferably from about 1:10 to about 1:60, preferably from about 1:10 to about 1:50, preferably from about 1:20 to about 1:40, preferably about 1:20, or preferably about 1:40.

In one or more embodiments, the skincare composition according to the present invention may further comprise an ingredient selected from the group consisting of surfactants, chelating agents, emollients, humectants, conditioners, preservatives, pearlescent or opacifiers, fragrances, and combinations of two or more thereof.

The skincare composition may include a surfactant, which may include nonionic, anionic, cationic, or amphoteric surfactants, or may include combinations of surfactants.

Examples of suitable nonionic surfactants include, but are not limited to, the fatty alcohol acid or amide ethoxylates, monoglyceride ethoxylates, sorbitan ester ethoxylates alkyl polyglycosides, and mixtures thereof.

Examples of anionic surfactants include salts of alkyl phosphate, glyceryl alkyl citrate, fatty acids in the anionic form, alkyl sulfate, alkyl sarcosinate, olefin sulfonate, alkyl taurate, or alkyl isethionate.

Examples of cationic surfactants that are suitable for use in this invention include alkyl quaternaries (mono, di, or tri), benzyl quaternaries, ester quaternaries, ethoxylated quaternaries, alkyl amines, and mixtures thereof, wherein the alkyl group has from about 6 carbon atoms to about 30 carbon atoms, with about 8 to about 22 carbon atoms being preferred.

Examples of amphoteric surfactants suitable for use in the present invention include, but are not limited to, amphocarboxylates such as alkylamphoacetates (mono or di); alkyl betaines; alkylamidoalkyl betaines (other than CAPB); alkylamidoalkyl sultaines; alkylamphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof.

The surfactant, or combination of surfactants, may be present in a total amount of from about 5% to about 30% by weight of the skincare composition, or from about 10% to about 20% by weight of the skincare composition.

The skincare composition may include an emollient, where the emollient or emollient blend is useful in cleansing the skin. As used herein, “emollients” refer to materials used for cleansing the skin, hair, and eye lashes, the prevention or relief of dryness, or for the protection of the skin. Examples of emollients useful herein include, but are not limited to, linear or branched chained hydrocarbon emollients, such as decane, isohexadecane, dodecane, tridecane, tetradecane, squalene, hexadecane, or isoparaffins; hydrophobic compounds such as vegetable oils; mineral oils (e.g., petrolatum); fatty esters (e.g., isopropyl palmitate, C12-C15 alkyl benzoate) including those fatty esters of glycerol, linear or branched alkyl esters; stearic esters, such as isostearates, including isopropyl isostearate, decyl isostearate (including simple isostearates and highly mono-branched decyl isostearates, which is an ester formed from an isostearic acid having an increased proportion of mono-branched molecules compared to simple isostearic acid), and hexyl isostearate; and combinations thereof.

The skincare composition may include one emollient or may include more than one emollient. The emollient(s) may be present in a total amount of from about 3% to about 15% by weight of the skincare composition, or from about 5% to about 10% by weight of the skincare composition.

The skincare composition may be free or substantially free of antimicrobial preservatives. The skincare composition may include antioxidants, such as tocopheryl acetate. If used, antioxidants may be present in an amount of from about 0.05% to about 0.5% by weight of the skincare composition, or from about 0.1% to about 0.25% by weight of the skincare composition.

The skincare composition may additionally include one or more humectants, such as panthenol or glycerin. Humectants, if used, may be present in an amount less than about 1% by weight, or less than about 0.5% by weight, or less than about 0.25% by weight.

The skincare composition may additionally include one or more fragrances or fragrance blends, including essential oils or other fragrance ingredients. Myrrh resin extract according to the invention is not considered as a fragrance. If used, fragrances are present in a total weight of from about 0.001% to about 0.5% by weight of the composition, or from about 0.01% to about 0.2% by weight of the composition. It may be preferred that the skincare composition be free or substantially free of added colorants or dyes. It may be preferred that the skincare composition be opaque and white, avoiding having a grayish color.

Examples of suitable pearlescent or opacifying agents include, but are not limited to mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO-(JO)a-H, wherein J is an alkylene group having from about 2 to about 3 carbon atoms; and a is 2 or 3; fatty alcohols containing from about 16 to about 22 carbon atoms; fatty esters of the formula: KCOOCH2L, wherein K and L independently contain from about 15 to about 21 carbon atoms; inorganic solids insoluble in the composition, and mixtures thereof.

In one or more embodiments, the skincare composition according to the present invention may be in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The skincare composition may be substantially free of retinoid compounds such as retinol.

As used herein, “essentially free” or “substantially free” of an ingredient means containing less than 0.1 weight percent, or less than 0.01 weight percent, or none of an ingredient.

Another aspect of the invention pertains to a skincare composition comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition.

The skincare composition may comprise:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition,
wherein the myrrh resin extract is obtained by extraction with an organic solvent, from the resin of a plant from the genus Commiphora.

The skincare composition may comprise:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition,
wherein the myrrh resin extract is obtained by extraction with an alcoholic solvent, from the resin of a plant from the genus Commiphora, and
wherein the weight ratio of myrrh resin extract to processed oat ingredient is from about 1:10 to about 1:80; preferably from about 1:10 to about 1:50, preferably from about 1:20 to about 1:40, preferably about 1:20, or preferably about 1:40.

The skincare composition may comprise:
a. a myrrh resin extract, from about 0.005 wt. % to about 1.5 wt. %, preferably from about 0.005% to about 0.1%, by total weight of the composition; and
b. a processed oat ingredient selected from the group consisting of oat oil, from about 0.1 wt. % to about 2.5 wt. %, by total weight of the composition, and
wherein the myrrh resin extract is obtained by extraction with an alcoholic solvent, from the resin of a plant from the genus Commiphora, and
wherein the weight ratio of myrrh resin extract to processed oat ingredient is from about 1:10 to about 1:80; preferably from about 1:10 to about 1:50, preferably from about 1:20 to about 1:40, preferably about 1:20, or preferably about 1:40, and
wherein the composition is substantially free of retinoid compounds such as retinol.

Another aspect of the invention pertains to a method of skin treatment comprising topically applying to the skin a skincare composition according to the present invention.

The method of skin treatment may be a cosmetic treatment of the skin.

Another aspect of the invention pertains to a method of treating sign of skin aging, comprising topically applying to the skin in need of treatment for skin aging a skincare composition according to the present invention.

The present invention is suitable for treating signs of skin aging. As used herein, “signs of skin aging” includes the presence of lines and wrinkles, loss of elasticity, uneven skin, and blotchiness. In a particularly preferred embodiment, the sign of aging is the presence of lines and wrinkles and/or loss of elasticity.

As used herein, “wrinkle” includes fine lines, fine wrinkles, or coarse wrinkles. Examples of wrinkles include, but are not limited to, fine lines around the eyes (e.g., “crow's feet”), forehead and cheek wrinkles, frown-lines, and laugh-lines around the mouth.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

The invention is also suitable for treating rosacea. As used herein, “rosacea” means skin with persistent erythema with or without papules, pustules, or nodules.

It has been observed that skincare compositions according to the present invention increase the production of ceramides in Normal Human Epidermal keratinocyte (NHEK) cells, and increases the expression of genes coding for skin barrier proteins. Therefore, a beneficial cosmetic effect by, for example, strengthening the barrier layer of the skin can be expected when applied topically on an aging skin.

In another aspect, the invention may pertain to a method of treating acne, comprising topically applying to the skin in need of treatment for acne a skincare composition according to the present invention. In a preferred embodiment the method of treating acne is a cosmetic method of acne treatment.

The invention is also suitable for treating acne. As used herein, “acne” refers to disorders resulting from the actions of hormones and other substances on the sebaceous glands and hair follicles, typically leading to clogged pores and the formation of inflammatory or non-inflammatory lesions on the skin. Specifically, it relates to blemishes, lesions, or pimples, pre-emergent pimples, blackheads, and/or whiteheads. As used herein, a “pre-emergent pimple” is an inflamed follicle that are not visually apparent on the surface of the skin with the naked eye (e.g., as a lesion).

In another aspect, the invention may pertain to a method of smoothing skin texture, comprising topically applying to the skin in need of treatment for smoother texture a skincare composition according to the present invention. In a preferred embodiment the method of smoothing skin texture is a cosmetic method of skin texture treatment.

In another aspect, the invention may pertain to a method of brightening the skin, comprising topically applying to the skin a skincare composition according to the present invention. In a preferred embodiment the method of brightening the skin is a cosmetic method of skin treatment.

Another aspect of the invention pertains to a method for activating lipid production in the skin layers of a skin in need thereof, comprising topically applying to the said skin a skincare composition comprising a myrrh resin extract and a processed oat ingredient.

In a preferred embodiment, the method for activating the lipid production in the skin layers of a skin in need thereof, according to the invention, is a cosmetic treatment method.

In another aspect, the invention may pertain to a skincare composition comprising a myrrh resin extract and a processed oat ingredient, for use in a cosmetic treatment method for activating the lipid production in the skin layers of a skin in need thereof.

Another aspect of the invention pertains to a method for activating the ceramide production in the skin layers of a skin in need thereof, comprising topically applying to the said skin a skincare composition comprising a myrrh resin extract and a processed oat ingredient.

In a preferred embodiment, the method for activating the ceramide production in the skin layers of a skin in need thereof, according to the invention, is a cosmetic treatment method.

In another aspect, the invention may pertain to a skincare composition comprising a myrrh resin extract and a processed oat ingredient, for use in a cosmetic treatment method for activating the ceramide production in the skin layers of a skin in need thereof.

In one or more embodiments, the method for activating the ceramide production comprises processed oat ingredient selected from the group consisting of fermented oat, colloidal oat, oat extract, oat oil, and combinations thereof.

In one or more embodiments, the method for activating the ceramide production comprises processed oat ingredient in an amount ranging from about 0.001% to about 10% by total weight of the composition.

Preferably the processed oat ingredient is present in an amount ranging from about 0.001% to about 5%; or from about 0.001% to about 1%; from about 0.001% to about 0.5%. Preferably the processed oat ingredient is present in an amount above 0.005% by total weight of the composition. Preferably the processed oat ingredient is present in an amount above 0.01% by total weight of the composition. Preferably the processed oat ingredient is present in an amount above 0.05% by total weight of the composition. Preferably the processed oat ingredient is present in an amount above 0.1% by total weight of the composition. Preferably the processed oat ingredient is present in an amount below 5% by total weight of the composition. Preferably the processed oat ingredient is present in an amount below 1% by total weight of the composition. Preferably the processed oat ingredient is present in an amount below 0.5% by total weight of the composition

In one or more embodiments, the method for activating the ceramide production comprises myrrh resin extract, wherein the myrrh resin extract is obtained from Commiphora Myrrha resin by extraction with an organic solvent.

In one or more embodiments, the method for activating the ceramide production comprises myrrh resin extract in an amount ranging from about 0.000001% to about 10% by total weight of the composition.

Preferably the myrrh resin extract is present in an amount ranging from about 0.000001% to about 1% by total weight of the composition; or about 0.000001% to about 0.5% by total weight of the composition; or about 0.000001% to about 0.2% by total weight of the composition; or about 0.000001% to about 0.1% by total weight of the composition; or about 0.000001% to about 0.01% by total weight of the composition, or about 0.000001% to about 0.02% by total weight of the composition, or about 0.00001% to about 0.02% by total weight of the composition, or about 0.0001% to about 0.02% by total weight of the composition, or about 0.001% to about 0.02% by total weight of the composition, or about 0.005% to about 1.5% by total weight of the composition, or about 0.005% to about 0.1% by total weight of the composition, or about 0.005% to about 0.015% by total weight of the composition. Preferably the myrrh resin extract is present in an amount above 0.001% by total weight of the composition. Preferably the myrrh resin extract is present in an amount above 0.005% by total weight of the composition. Preferably the myrrh resin extract is present in an amount above 0.01% by total weight of the composition. Preferably the myrrh resin extract is present in an amount below 1% by total weight of the composition. Preferably the myrrh resin extract is present in an amount below 0.5% by total weight of the composition. Preferably the myrrh resin extract is present in an amount below 0.2% by total weight of the composition. Preferably the myrrh resin extract is present in an amount below 0.1% by total weight of the composition. Preferably the myrrh resin extract is present in an amount below 0.05% by total weight of the composition. Preferably the myrrh resin extract is present in an amount below 0.02% by total weight of the composition.

In one or more embodiments, the method for activating the ceramide production comprises topically applying a skincare composition comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition.

Preferably the method for activating the ceramide production may comprise topically applying a skincare composition comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition, wherein the myrrh resin extract is obtained by extraction with an organic solvent, from the resin of a plant from the genus Commiphora.

Preferably the method for activating the ceramide production may comprise topically applying a skincare composition comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition,
wherein the myrrh resin extract is obtained by extraction with an alcoholic solvent, from the resin of a plant from the genus Commiphora, and
wherein the weight ratio of myrrh resin extract to processed oat ingredient is from about 1:10 to about 1:80; preferably from about 1:10 to about 1:60, preferably from about 1:20 to about 1:50, preferably from about 1:30 to about 1:50, preferably about 1:40.

In one or more embodiments, the method for activating the ceramide production comprises a skincare composition according to the present invention, wherein the skincare composition is substantially free of retinoid compounds such as retinol.

Preferably the method for activating the ceramide production may comprise topically applying a skincare composition comprising:
a. a myrrh resin extract, from about 0.005 wt. % to about 1.5 wt. %, preferably from 0.005% to about 0.1%, by total weight of the composition; and
b. a processed oat ingredient selected from the group consisting of oat oil, from about 0.1 wt. % to about 2.5 wt. %, by total weight of the composition, and
wherein the myrrh resin extract is obtained by extraction with an alcoholic solvent, from the resin of a plant from the genus Commiphora, and
wherein the weight ratio of myrrh resin extract to processed oat ingredient is from about 1:10 to about 1:80; preferably from about 1:10 to about 1:60, preferably from about 1:20 to about 1:50, preferably from about 1:30 to about 1:50, preferably about 1:40, and
wherein the composition is substantially free of retinoid compounds such as retinol.

Another aspect of the invention pertains to a method for boosting skin Barrier by stimulating skin barrier genes expression, for example Involucrin, Filaggrin and Loricrin expressions, in the skin layers of a skin in need thereof, comprising topically applying to the said skin a skincare composition comprising a myrrh resin extract and a processed oat ingredient.

By “boosting skin barrier” it is meant increasing the natural protection conferred by the skin barrier to the organism, which is to prevent trans epidermal water loss and to protect the organism from external aggression such as biological (Microorganisms), chemical, environmental.

In a preferred embodiment, the method for boosting skin Barrier by stimulating skin barrier genes expression in the skin layers of a skin in need thereof, according to the invention, is a cosmetic treatment method.

Another aspect of the invention pertains to a method for boosting the anti-aging effects of processed oat in the skin layers of a skin in need thereof, comprising topically applying to skin a skincare composition comprising a myrrh resin extract and a processed oat ingredient, preferably selected from the group consisting of fermented oat, colloidal oat, oat extract, oat oil, and combinations thereof.

By “boosting the anti-aging effects” it is meant that the signs of skin aging are reduced and less visible.

In a preferred embodiment, the method for boosting the anti-aging effects of processed oat in the skin layers of a skin in need thereof, according to the invention, is a cosmetic treatment method.

In another aspect, the invention may pertain to a skincare composition comprising a myrrh resin extract and a processed oat ingredient, for use in a cosmetic treatment method for activating the barrier proteins production in the skin layers of a skin in need thereof.

Another aspect of the invention pertains to a method for activating the barrier proteins production in the skin layers of a skin in need thereof, comprising topically applying to the said skin a skincare composition comprising a myrrh resin extract and a processed oat ingredient.

In a preferred embodiment, the method for activating the barrier proteins production in the skin layers of a skin in need thereof, according to the invention, is a cosmetic treatment method.

In another aspect, the invention may pertain to a skincare composition comprising a myrrh resin extract and a processed oat ingredient, for use in a cosmetic treatment method for activating the barrier proteins production in the skin layers of a skin in need thereof.

In one or more embodiments, the method for activating the barrier proteins production comprises processed oat ingredient selected from the group consisting of fermented oat, colloidal oat, oat extract, oat oil, and combinations thereof.

In one or more embodiments, the method for activating the barrier proteins production comprises processed oat ingredient in an amount ranging from about 0.001% to about 10% by total weight of the composition.

Preferably the processed oat ingredient is present in an amount ranging from about 0.001% to about 5%; or from about 0.001% to about 1%; from about 0.001% to about 0.5%. Preferably the processed oat ingredient is present in an amount above 0.005% by total weight of the composition. Preferably the processed oat ingredient is present in an amount above 0.01% by total weight of the composition. Preferably the processed oat ingredient is present in an amount above 0.05% by total weight of the composition. Preferably the processed oat ingredient is present in an amount above 0.1% by total weight of the composition. Preferably the processed oat ingredient is present in an amount below 5% by total weight of the composition. Preferably the processed oat ingredient is present in an amount below 1% by total weight of the composition. Preferably the processed oat ingredient is present in an amount below 0.5% by total weight of the composition

In one or more embodiments, the method for activating the barrier proteins production comprises myrrh resin extract, wherein the myrrh resin extract is obtained from Commiphora Myrrha resin by extraction with an organic solvent.

In one or more embodiments, the method for activating the barrier proteins production comprises myrrh resin extract in an amount ranging from about 0.000001% to about 10% by total weight of the composition.

Preferably the myrrh resin extract is present in an amount ranging from about 0.000001% to about 1% by total weight of the composition; or about 0.000001% to about 0.5% by total weight of the composition; or about 0.000001% to about 0.2% by total weight of the composition; or about 0.000001% to about 0.1% by total weight of the composition; or about 0.000001% to about 0.01% by total weight of the composition, or about 0.000001% to about 0.02% by total weight of the composition, or about 0.00001% to about 0.02% by total weight of the composition, or about 0.0001% to about 0.02% by total weight of the composition, or about 0.001% to about 0.02% by total weight of the composition, or about 0.005% to about 1.5% by total weight of the composition, or about 0.005% to about 0.1% by total weight of the composition, or about 0.005% to about 0.015% by total weight of the composition. Preferably the myrrh resin extract is present in an amount above 0.001% by total weight of the composition. Preferably the myrrh resin extract is present in an amount above 0.005% by total weight of the composition. Preferably the myrrh resin extract is present in an amount above 0.01% by total weight of the composition. Preferably the myrrh resin extract is present in an amount below 1% by total weight of the composition. Preferably the myrrh resin extract is present in an amount below 0.5% by total weight of the composition. Preferably the myrrh resin extract is present in an amount below 0.2% by total weight of the composition. Preferably the myrrh resin extract is present in an amount below 0.1% by total weight of the composition. Preferably the myrrh resin extract is present in an amount below 0.05% by total weight of the composition. Preferably the myrrh resin extract is present in an amount below 0.02% by total weight of the composition.

In one or more embodiments, the method for activating the barrier proteins production comprises topically applying a skincare composition comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition.

Preferably the method for activating the barrier proteins production may comprise topically applying a skincare composition comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition, wherein the myrrh resin extract is obtained by extraction with an organic solvent, from the resin of a plant from the genus Commiphora.

Preferably the method for activating the barrier proteins production may comprise topically applying a skincare composition comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition,
wherein the myrrh resin extract is obtained by extraction with an alcoholic solvent, from the resin of a plant from the genus Commiphora, and
wherein the weight ratio of myrrh resin extract to processed oat ingredient is from about 1:10 to about 1:80; preferably from about 1:10 to about 1:50, preferably from about 1:20 to about 1:40, preferably about 1:20, or preferably about 1:40.

In one or more embodiments, the method for activating the barrier proteins production comprises a skincare composition according to the present invention, wherein the skincare composition is substantially free of retinoid compounds such as retinol.

Preferably the method for activating the barrier proteins production may comprise topically applying a skincare composition comprising:
a. a myrrh resin extract, from about 0.005 wt. % to about 1.5 wt. %, preferably from 0.005% to about 0.1%, by total weight of the composition; and
b. a processed oat ingredient selected from the group consisting of oat oil, from about 0.1 wt. % to about 2.5 wt. %, by total weight of the composition, and
wherein the myrrh resin extract is obtained by extraction with an alcoholic solvent, from the resin of a plant from the genus Commiphora, and
wherein the weight ratio of myrrh resin extract to processed oat ingredient is from about 1:10 to about 1:80; preferably from about 1:10 to about 1:50, preferably from about 1:20 to about 1:40, preferably about 1:20, or preferably about 1:40, and
wherein the composition is substantially free of retinoid compounds such as retinol.

The present invention may be better understood through the following examples, which are exemplary in nature and not intended to be limiting to any specific combination of elements.

### Examples

Myrrh resin extract (Commiphora Myrrha resin extract) was supplied by Firmenich.

Oat oil was supplied by Symrise, Teterboro, NJ.

### Example 1-Ceramides Production

### Methods

1 vial of 10 donor pooled Normal Human Epidermal keratinocyte (NHEK) cells was seeded into a T-175 flask with 35mls of complete Keratinocyte growth media (KGM) from Lifeline Cell Technology, Part #LM-004. The flask containing the cells and the media was then placed into a tissue culture incubator and kept at 37° C. and 5% CO₂. Four days after incubation the seeded flask had a keratinocyte cell density around 80% confluency.

All media was then removed from the flask and the cells were twice rinsed with Phosphate Buffered Saline (PBS) solution. All PBS was then removed and 5 mls of Trypsin/EDTA was added to the flask and allowed to rest on the NHEK cells in the incubator for 5 mins to detach the NHEK cells from the flask.

5 mls of Trypsin Neutralizing solution (TNS) was then added to the flask and the detached cells and solution were then transferred to a 15 ml sterile conical tube which was subsequently centrifuged for 5 mins at 125G. At the end of the centrifugation, all supernatant was removed leaving the keratinocyte pellet at the bottom of the tube intact and undisturbed.

10 mls of fresh KGM was then added to the tube containing the pellet and used to resuspend the NHEK pellet. The resuspended NHEK cells were then counted and used to seed 4 6-well plates with 2mls of KGM per well at an NHEK cell density of 2.5×10⁵ cells/well. The 4 6-well plates were then placed in the incubator for 2 days after which time the cells were 100% confluent in each well. All media was then removed and replaced with 2mls of triplicate media or treatment solutions and 2.4 mM CaCl₂) solution.

Treatment solutions were Myrrh resin extract and/or Oat oil dissolved in DMSO and diluted in media (KGM) to give final treatment doses of 10 ppm or 20 ppm Myrrh resin extract, and 200 ppm or 400 ppm Oat oil, or combination: 10 ppm Myrrh resin extract and 200 ppm Oat oil, 10 ppm Myrrh resin extract and 400 ppm Oat oil.

Three days after initial treatment all treatments were removed and replaced with 2mls of fresh and similar treatments and the plates were then returned to the incubator. Two days after the second treatment was added the plates were removed from the incubator and all media was removed from the wells and each well was rinsed 2 times with 1 ml of cold PBS. All PBS was removed after the second rinse and 1 ml of fresh PBS was added to each well.

The NHEK cells in each well was scraped and the PBS/NHEK solution was transferred to a pre-weighed, appropriately labeled Eppendorf tube. The Eppendorf tubes were then spun at 13,200 RPM for 15 mins, all supernatant was then removed from each Eppendorf tube and the remaining pellet was then weighed and frozen at −15° C. until transferred to Analytical for analysis of ceramide content and value.

### Analysis

### Sample Extraction:

1. 1 ml of Extraction Solvent (EtOH: EtOAc, 1:1) was added into each sample tube (samples were kept in 1.5 ml of micro centrifuge tube separately), sonicated 30 minutes.

2. Centrifuged 10 min at 13,000 rpm.

3. For Keratinocyte tissue sample, the supernatant was transferred into HPLC vial for HPLC-MS analysis.

### UPLC-Q TOF MS Analysis:

### UPLC Conditions

Mobile Phase A: 5 mM ammonium formate in water
Mobile Phase B: MeOH/i-PrOH (90:10, v/v)
Needle Rinse: MeOH/i-PrOH (50:50, v/v)
HPLC column: Waters, Acquity UPLC BEH C8, 2.1×100 mm, 1.7 μm
Column temperature: 45° C.
Injection volume: 2 μl for positive and 4 μl for negative

Binary gradient elution was as indicated in table 1 below:

TABLE 1

| Time (min) | Flow (ml/min) | A (%) | B (%) |
|---|---|---|---|
| 0.0 | 0.4 | 30.0 | 70.0 |
| 1.0 | 0.4 | 30.0 | 70.0 |
| 10.0 | 0.4 | 2.0 | 98.0 |
| 15.0 | 0.4 | 2.0 | 98.0 |
| 18.0 | 0.4 | 30.0 | 70.0 |
| 20.0 | 0.4 | 30.0 | 70.0 |

MS Conditions were as listed in table 2 below:

TABLE 2

| MS Parameters | Settings |
|---|---|
| System | Agilent 1290/Agilent 6545 Q TOF MS |
| Ionization source | Dual AJS ESI, positive and negative modes |
| Calibration | Low (1,700); 4 GHz High Resolution |
| | Scan Range: 300-1,700 m/z (centroid) |
| Gas Temp | 325° C. |
| Drying Gas | 12 L/min |
| Nebulizer | 35 psig |
| Sheath Temp | 350° C. |
| Sheath Gas Flow | 11 L/min |
| Vcap | 3500 V |
| Nozzle Voltage (Expt) | 2,000 V |
| Fragmentor | 150 V |
| Skimmer | 65 V |
| Oct 1RF Vpp | 750 V |

### Data Process:

Generate 500 highest MS peaks using Find Molecule Feature (FMF) from negative ionization.

Search the data base from on-line open-source LIPID MAPS® Website for ceramides match.

Remove all unmatched peaks and sum the total peak responses for ceramides.

From positive ionization, use 1-stearroyl-2-linoleoyl-sn-glycero-3-phosphatidylcholine (SLPC) peak area (m/z 786.606) as the marker for tissue normalization.

Report final normalized total ceramide results.

In the tables 3 to 6 below the “Actual increase” indicates the % of increase measured according to the protocol described above. For example, a 100% increase of ceramide means that the production of ceramide has been multiplied by 2.

“Expected increase” refers to the anticipated increase for a combination of ingredients based on the observed increases when the ingredients are taken separately. For example, if both ingredients were measured to increase the production of ceramide by 100% (2 fold), then the combination of the two would be expected to increase the production of ceramide by 200% (3 fold).

“% synergy” refers to the difference observed, for combination of Myrrh resin extract and Oat oil, between the measured increase and the expected increase. Difference is then divided by the expected increase value and multiplied by 100 to obtain a percentage.

$$Synergy ( \% ) = [ ( Actual ( \% ) - Expected ( \% ) ) / Expected ( \% ) ] * 100$$

If the measured increase is superior to what could have been expected based on the observed increases when the ingredients are taken separately, then a positive synergy is observed, and the % synergy value is positive.

In the tables 3 to 6 below, the treatment with “Myrrh” is an abbreviation and refers to a Myrrh resin extract according to the invention.

### Results

TABLE 3
Ceramides

| | Actual | Expected | % |
|---|---|---|---|
| Treatments | increase ** | increase | synergy |
| Myrrh 10 ppm | 109% | | |
| Oat Oil 200 ppm | 95% | | |
| Myrrh 10 ppm + Oat oil 200 ppm | 106% | 204% | −48.04 |
| Myrrh 10 ppm | 109% | | |
| Oat oil 400 ppm | 111% | | |
| Myrrh 10 ppm + Oat oil 400 ppm | 381% | 220% | 73.18 |
| Myrrh 20 ppm | 197% | | |

** (result versus the untreated control values)

Combination of 10 ppm Myrrh resin extract and 200 ppm Oat Oil doesn't lead to any increase in ceramide production when compared to 10 ppm Myrrh resin extract alone or 200 ppm Oat Oil alone. No cumulative effect was observed at this dosage.

Surprisingly, it was observed that a combination of 10 ppm Myrrh resin extract and 400 ppm Oat Oil provided an unexpectedly high production of ceramide. The expected cumulative effect of 10 ppm Myrrh resin extract and 400 ppm Oat Oil was around 220%, slightly above a 3 fold increase; but the measured values were much higher, about 380%, almost a 5 fold increase in ceramide production.

This unexpected result is a 73% synergy, meaning the observed value are 1.73 fold higher than what could have been expected based on a cumulative effect. This effect is particularly spectacular considering the negative results obtained with 10 ppm Myrrh resin extract and 200 ppm Oat Oil.

Experiments were also carried out with a 20 ppm Myrrh resin extract. Although the ceramide production increased accordingly, this dosage appeared to give negative results regarding toxicology. Further investigations were thus not conducted with 20 ppm Myrrh resin extract.

Table 4 provides results for the same set of experiments as disclosed in table 3 except that the results are expressed in ceramide production normalized according to the weight of collected material (remaining pellet as disclosed in the protocol detailed above).

TABLE 4
Ceramides/weight

| | Actual | Expected | % |
|---|---|---|---|
| Treatments | increase ** | increase | synergy |
| Myrrh 10 ppm | 195% | | |
| Oat Oil 200 ppm | 199% | | |
| Myrrh 10 ppm + Oat oil 200 ppm | 249% | 394% | −36.80 |
| Myrrh 10 ppm | 195% | | |
| Oat oil 400 ppm | 247% | | |
| Myrrh 10 ppm + Oat oil 400 ppm | 563% | 442% | 27.38 |
| Myrrh 20 ppm | 480% | | |

** (result versus the untreated control values)

Tables 5 and 6 provide results for the same set of experiments as disclosed in table 3 except that the results are expressed in ceramide production normalized according to other lipids produced in the same sample, DPPC for table 5, and SLPC for table 6. DPPC and SLPC are used as internal references.
DPPC means: Dipalmitoylphosphatidylcholine
SLPC means: 1-stearroyl-2-linoleoyl-sn-glycero-3-phosphatidylcholine

TABLE 5
Ceramides/DPPC

| | Actual | Expected | % |
|---|---|---|---|
| Treatments | increase ** | increase | synergy |
| Myrrh 10 ppm | 141% | | |
| Oat Oil 200 ppm | 96% | | |
| Myrrh 10 ppm + Oat oil 200 ppm | 196% | 237% | −17.46 |
| Myrrh 10 ppm | 141% | | |
| Oat oil 400 ppm | 153% | | |
| Myrrh 10 ppm + Oat oil 400 ppm | 749% | 294% | 154.76 |
| Myrrh 20 ppm | 327% | | |

** (result versus the untreated control values)

TABLE 6
Ceramides/SLPC

| | Actual | Expected | % |
|---|---|---|---|
| Treatments | increase ** | increase | synergy |
| Myrrh 10 ppm | 111% | | |
| Oat Oil 200 ppm | 89% | | |
| Myrrh 10 ppm + Oat oil 200 ppm | 139% | 200% | −30.50 |
| Myrrh 10 ppm | 111% | | |
| Oat oil 400 ppm | 135% | | |
| Myrrh 10 ppm + Oat oil 400 ppm | 562% | 246% | 128.46 |
| Myrrh 20 ppm | 234% | | |

** (result versus the untreated control values)

A similar trend to the results presented in table 3 is observed for the results presented in tables 4, 5 and 6.

10 ppm Myrrh resin extract and 200 ppm Oat oil don't lead to any improvement and the results are below what could have been expected from a cumulative effect, whereas the results for 10 ppm Myrrh resin extract and 400 ppm Oat oil are superior to cumulative effect and demonstrate a synergy.

### Example 2-Barrier Gene Expression

### Methods

1 vial of 10 donor Normal Human Epidermal keratinocyte (NHEK) cells was thawed and seeded into a T175 flask with 35 mls of complete Keratinocyte growth media (KGM). The seeded flask was then incubated at 37 degrees Celsius and 5% CO₂ for 4 days. At day 4 the 85% confluent flask was rinsed once with 6mls of Dulbecco Phosphate Buffered Saline solution (DPBS) and the cells were detached by adding 4mls of 0.05% trypsin/EDTA for 3 mins, after which time 4mls of Trypsin neutralizing solution (TNS) was added to the flask.

The detached cells and solution were then transferred to a 15 ml tube and spun at 125 G for 5 mins. All supernatant was then removed from the 15 ml tube and 10 mls of fresh, complete KGM was used to resuspend the remaining pellet of cells. The resuspended cells were then counted and adjusted to a concentration of 1.8×10⁵ cells/ml. A total of 50mls of cell suspension was prepared and 1 ml of the cell suspension was added to each well of the 12-well plates. A total of 4 12-well plates were seeded. 24 hrs after seeding the NHEK cells into 4 12-well plates each well was 95% confluent. At that time all media in each well was removed and replaced with the relevant treatments in media solution and the treated plates were returned to the incubator for a period of 72 hours.

Treatment solutions were Myrrh resin extract and/or Oat oil dissolved in DMSO and diluted in media (KGM) to give final treatment doses of 10 ppm or 20 ppm Myrrh resin extract, and 200 ppm or 400 ppm Oat oil, or combination: 10 ppm Myrrh resin extract and 200 ppm Oat oil, 10 ppm Myrrh resin extract and 400 ppm Oat oil, 20 ppm Myrrh resin extract and 200 ppm Oat oil, 20 ppm Myrrh resin extract and 400 ppm Oat oil.

At the end of 72 hours all media was removed from the wells and replaced with 350 μl of RLT buffer+0.001% Mercaptoethanol. From this point onward RNAse free ware and techniques were used; the cells were scraped from the bottom of each well and the cells and the lysis buffer solution were then transferred to appropriately labeled Eppendorf tubes. The Eppendorf tubes and their content were then transferred to a −80° C. freezer and stored until the RNA purification step.

RNA was extracted from the solutions using the RNEASY Mini Kit (Qiagen, Valencia, CA) and according to manufacturer's instructions and RNA was eluted in 30 μL RNase-free water. Reverse transcription (RT) was performed using the Invitrogen SuperScript IV ViLo kit (ThermoFisher Scientific, Bridgewater, NJ).

Gene expression was performed using the gene expression assays sold under the tradename TAQMAN for Keratinocyte skin Barrier related genes, Loricrin, Filaggrin and Involucrin and polymerase (RNA) II polypeptide A (POLR2A), and fast advanced master mix sold under the tradename TAQMAN (ThermoFisher Scientific, Bridgewater, NJ). qPCR analysis was performed using the TAQMAN fast advanced master mix and run on a real time PCR system sold under the tradename QUANTSTUDIO 7 Flex System (ThermoFisher Scientific, Bridgewater, NJ).

The expression of these genes was normalized against the expression of the human POLR2A “housekeeping” gene. This “housekeeping” gene is a widely used control for normalizing specific gene expression levels because of their invariant expression across tissues, cells, and experimental treatments. The method for normalization involves measuring the expression of an internal reference or “housekeeping” gene, which considers the potential error of RNA/cDNA loading, and variation of reverse transcription efficiency.

The increases in expression were calculated in comparison to the untreated and two-tailed two-sample Student t-tests (Microsoft Office Excel 2007; Microsoft, Redmond, WA, USA) were performed.

Results for Fillagrin (table 7), Involucrin (table 8) and Loricrin (table 9) are shown as percentage of expression increase over untreated in Tables 7-9 below.

“Untreated” means that the gene expression assays were performed with only media added to the Keratinocyte cells.

In the tables 7 to 9 below the “Actual increase” indicates the % of increase measured according to the protocol described above. For example, a 100% increase of barrier gene expression means that the expression of barrier gene has been multiplied by 2.

“Expected increase” refers to the anticipated increase for a combination of ingredients based on the observed increases when the ingredients are taken separately. For example, if both ingredients were measured to increase the expression of barrier gene by 100% (2 fold), then the combination of the two would be expected to increase the expression of barrier gene by 200% (3 fold).

“% synergy” refers to the difference observed, for combination of Myrrh resin extract and Oat oil, between the measured increase and the expected increase. Difference is then divided by the expected increase value and multiplied by 100 to obtain a percentage.

$$Synergy ( \% ) = [ ( Actual ( \% ) - Expected ( \% ) ) / Expected ( \% ) ] * 100$$

If the measured increase is superior to what could have been expected based on the observed increases when the ingredients are taken separately, then a positive synergy is observed, and the % synergy value is positive.

In the tables 7 to 9 below, the treatment with “Myrrh” is an abbreviation and refers to a Myrrh resin extract according to the invention.

### Results

TABLE 7
Fillagrin gene expression

| | Actual | Expected | % |
|---|---|---|---|
| Fillagrin | increase ** | increase | synergy |
| Myrrh 10 ppm | 37% | | |
| Oat Oil 200 ppm | 301% | | |
| Myrrh 20 ppm | 550% | | |
| Oat Oil 400 ppm | 171% | | |
| Myrrh 10 ppm + Oat Oil 200 ppm | 593% | 338% | 75% |
| Myrrh 10 ppm + Oat Oil 400 ppm | 603% | 208% | 190% |
| Myrrh 20 ppm + Oat Oil 200 ppm | 571% | 851% | −33% |
| Myrrh 20 ppm + Oat Oil 400 ppm | 474% | 721% | −34% |

** (result versus the untreated control values)

TABLE 8
Involucrin gene expression

| | Actual | Expected | % |
|---|---|---|---|
| Involucrin | increase ** | increase | synergy |
| Myrrh 10 ppm | 139% | | |
| Oat Oil 200 ppm | 174% | | |
| Myrrh 20 ppm | 2206% | | |
| Oat Oil 400 ppm | 70% | | |
| Myrrh 10 ppm + Oat Oil 200 ppm | 550% | 313% | 76% |
| Myrrh 10 ppm + Oat Oil 400 ppm | 491% | 209% | 135% |
| Myrrh 20 ppm + Oat Oil 200 ppm | 1566% | 2380% | −34% |
| Myrrh 20 ppm + Oat Oil 400 ppm | 1956% | 2276% | −14% |

** (result versus the untreated control values)

TABLE 9
Loricrin gene expression

| | Actual | Expected | % |
|---|---|---|---|
| Loricrin | increase ** | increase | synergy |
| Myrrh 10 ppm | 42% | | |
| Oat Oil 200 ppm | 519% | | |
| Myrrh 20 ppm | 1475% | | |
| Oat Oil 400 ppm | 210% | | |
| Myrrh 10 ppm + Oat Oil 200 ppm | 1172% | 561% | 109% |
| Myrrh 10 ppm + Oat Oil 400 ppm | 1196% | 252% | 375% |
| Myrrh 20 ppm + Oat Oil 200 ppm | 1178% | 1994% | −41% |
| Myrrh 20 ppm + Oat Oil 400 ppm | 1853% | 1685% | 10% |

** (result versus the untreated control values)

Combination of 10 ppm Myrrh resin extract and 200 ppm or 400 ppm Oat Oil led to any increase in barrier gene expression when compared to 10 ppm Myrrh resin extract alone or 200 ppm/400 ppm Oat Oil alone.

Surprisingly, it was observed that a combination of 10 ppm Myrrh resin extract and 200 ppm or 400 ppm Oat Oil provided an unexpectedly high barrier gene expression. Synergistic effects are observed for both 10 ppm Myrrh resin extract and 200 ppm Oat Oil; and 10 ppm Myrrh resin extract and 400 ppm Oat Oil.

As observed in Example 1 on Ceramide production, 10 ppm Myrrh resin extract and 400 ppm Oat Oil provides a strong synergy. Interestingly when compared to Example 1, 10 ppm Myrrh resin extract and 200 ppm Oat Oil also provides a synergy even if inferior to the effect observed for 10 ppm Myrrh resin extract and 400 ppm Oat Oil. This suggests that an unexpected beneficial effect to improve skin barrier by enhancing skin's natural protein synthesis from within may be achieved.

Experiments were also carried out with a 20 ppm Myrrh resin extract alone or in combination with 200 ppm or 400 ppm Oat Oil. Increases in the barrier gene expressions were observed; however no, or very low, synergistic effects were observed.

### Conclusion

Myrrh extract alone induced epidermal ceramides and barrier gene expression, and this pro-ceramide and barrier related genes benefit was further increased in combination with oat oil.

When barrier related genes such as Involucrin, Filaggrin and Loricrin are upregulated a cascade of barrier related proteins are also upregulated and the skin's protective barrier is believed to be enhanced.

Myrrh resin extract and oat oil combinations thereby provides an opportunity to improve from within the skin barrier by enhancing the production of skin natural ceramide and barrier related proteins.

It has been observed that an active dose of a compound, for example myrrh resin extract or retinol, providing an in-vitro benefit may generally requires a 10-fold increase in-vivo to demonstrate similar benefits. A 10 ppm dose corresponds to 0.001% and thus a 10-fold increase (100 ppm) corresponds to 0.01%.

1. A skincare composition comprising:
a. a myrrh resin extract, and
b. a processed oat ingredient.

2. The skincare composition of claim 1, wherein the myrrh resin extract is produced from the resin of a plant from the genus Commiphora.

3. The skincare composition of claim 2, wherein the myrrh resin extract is obtained by extraction with an organic solvent.

4. The skincare composition according to claim 1, wherein the myrrh resin extract is present in an amount ranging from about 0.000001% to about 10% by total weight of the composition.

5. The skincare composition according to claim 1, wherein the processed oat ingredient is selected from the group consisting of fermented oat, colloidal oat, oat extract, oat oil, and combinations thereof.

6. The skincare composition according to claim 1, wherein the processed oat ingredient is present in an amount ranging from about 0.001% to about 10% by total weight of the composition.

7. The skincare composition according to claim 1, wherein the weight ratio of myrrh resin extract to processed oat ingredient is from about 10:1 to about 1:1000.

8. The skincare composition according to claim 1, further comprising an ingredient selected from the group consisting of surfactants, chelating agents, emollients, humectants, conditioners, preservatives, opacifiers, fragrances, and combinations of two or more thereof.

9. The skincare composition according to claim 1, wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

10. The skincare composition of claim 1, comprising:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition.

11. A method of skin treatment comprising topically applying to the skin a composition according to claim 1.

12. A method of treating sign of skin aging, comprising topically applying to the skin in need of treatment for skin aging a skincare composition according to claim 1.

13. A method for activating lipid production in the skin layers of a skin in need thereof, comprising topically applying to the said skin a skincare composition according to claim 1.

14. A method for activating ceramide production in the skin layers of a skin in need thereof, comprising topically applying to the said skin a skincare composition according to claim 1.

15. The method according to claim 14, wherein the processed oat ingredient is selected from the group consisting of fermented oat, colloidal oat, oat extract, oat oil, and combinations thereof.

16. The method according to claim 14, wherein the processed oat ingredient is present in an amount ranging from about 0.001% to about 10% by total weight of the skincare composition.

17. The method according to claim 14, wherein the myrrh resin extract is present in an amount ranging from about 0.000001% to about 10% by total weight of the composition.

18. The method according to claim 14, wherein the skincare composition topically applied to the skin comprises:
a. a myrrh resin extract, from about 0.005 wt. % to about 1.5 wt. %, by total weight of the composition; and
b. a processed oat ingredient selected from the group consisting of oat oil, from about 0.1 wt. % to about 2.5 wt. %, by total weight of the composition.

19. A method for boosting skin Barrier by stimulating skin barrier genes expression, for example Involucrin, Filaggrin and Loricrin expressions, in the skin layers of a skin in need thereof, comprising topically applying to the said skin a skincare composition according to claim 1.

20. A method for boosting the anti-aging effects of processed oat in the skin layers of a skin in need thereof, comprising topically applying to skin a skincare composition according to claim 1.
